(19) United States
(12) Patent Application Publication
(10) Pub. No.: US 2007/0243563 A1
(43) Pub. Date: Oct. 18, 2007

(54) Aflatoxin-albumin adduct measurement and the uses thereof

(75) Inventors: Mei-Hui Wang, North District Hsin Chu (TW); Chang-Yi Wu, Taipei (TW); Te-Wei Lee, Taipei (TW); Henton Huang, Tau Yen (TW); Gann Ting, Taipei (TW)

Correspondence Address: APEX JURIS, PLLC;TRACY M HEIMS, LAKE CITY CENTER, SUITE 410, 12360 LAKE CITY WAY NORTHEAST, SEATTLE, WA, 98125, US

(73) Assignee: Institute of Nuclear Energy Research, Tau Yen (TW)

(21) Appl. No.: 11/236,991

(22) Filed: Sep. 28, 2005

Related U.S. Application Data
(63) Continuation-in-part of application No. 10/041,478, filed on Jan. 10, 2002, now abandoned.
Application No. 10/041,478 is a division of application No. 09/718,341, filed on Nov. 24, 2000, now abandoned.

(51) Int. Cl. G01N 33/574; G01N 33/543

(52) U.S. Cl. 435/7.23; 436/518

(57) ABSTRACT

The invention discloses one immunoassay for aflatoxin-albumin adducts measurement in serum. In Particular, serum pretreatments including albumin precipitation, resuspension, dialysis, albumin digestion, enzyme precipitation and aflatoxin extraction, centrifugation and resuspension are unnecessary, which is convenient for clinical routine serum testing. The invention also discloses their clinical uses in rapid measurement of the doses of aflatoxin exposure, which is one of risk factors of liver cancer.

### CROSS REFERENCE TO RELATED APPLICATIONS

This is a continuation-in-part of Ser. No. 10/041,478, filed on Jan. 10, 2002, pending, which is a divisional of Ser. No. 09/718,341, filed on Nov. 24, 2000, now abandoned.

### FIELD OF THE INVENTION

The present invention relates to an aflatoxin-albumin adduct measurement. In particular, the present invention relates to routine uses with serum. The present invention also relates to a method for evaluation aflatoxin exposure. The normal cutoff value is also disclosed.

### BACKGROUND OF THE INVENTION

Aflatoxins are toxic metabolites produced by the fungal species Aspergillus flavus and Aspergillus parasiticus. Aflatoxin B1 (AFB1) is the most toxic group. Experimental evidence suggests that aflatoxin is hepatotoxic as well as carcinogenic, especially as regards hepatocellular carcinomas. Using the TD50 values for rats developed by Gold et al (Cancer Res. 1993, 53: 9-11), AFB1, which TD50=9.3×10⁻⁴ mg/kg per day, is 1000 times more potent a carcinogen than benzo(a)pyrene. Recently, the International Agency for Research on Cancer (IARC) reported that there is sufficient evidence to classify aflatoxin B1 and mixtures of aflatoxins as Group 1 carcinogens in humans.

AFB1 requires microsomal oxidation to the reactive AFB1-8,9-epoxide (AFBO) to exert its hepatocarcinogenic effects, and for the covalent binding of AFBO to cellular RNA, DNA, protein or other macromolecules (IARC 1993;56:303).

In 1987, Sabbioni et al. reported excreted RNA and DNA adducts only reflect the previous 24-48 hours of exposure. A protein-based dosimeter which reflects weeks of exposure history could add greatly to the interpretation of epidemiological data.

Hemoglobin has the longest biological half-life among the proteins common proposed for dosimetry, but it binds to AFB1 very low. Serum albumin, in contrast, binds a large fraction of ingested AFB1 as a stable covalent adduct. In humans, the half-life for turnover of serum albumin is about 20 days, which leads to chronic exposure and adduct levels reflecting exposure during the past 1-2 months (Carcinogenesis 8: 819-824,1987)

In prior art, methods for testing of aflatoxin-albumin adduct in serum are the same as those for testing of aflatoxin before 1996. The serum sample needs to be digested with a protease and extracted in organic solvent to get pure aflatoxin, and then measured aflatoxin with prior art such as high performance liquid chromatography, radioimmunoassay, or enzyme-linked immunosorbent assay. The pretreatment, that is, of the process of extraction and purification, is tedious. It is not convenient for clinical routine usage. The pretreatment of serum is referred to in Chen Chien-Jen et al. (1996). Elevated Aflatoxin Exposure and Increased Risk of Hepatocellular Carcinoma. Hepatology, 24(1), 38-42, and includes albumin determination, protein precipitation, resuspension and dialysis, as well as protein digestion, enzyme precipitation and aflatoxin extraction and finally centrifugation and resuspension. In Wang L Y et al., (1996) Alflatoxin Exposure and Risk of Hepatocellular Carcinoma in Taiwan. Int. J. Cancer, 67, 620-625, serum pretreatment is described to include protein digestion with protein kinase K from Boehringer Mannheim, Indianapolis, Ind. Furthermore, serum pretreatment included concentration, filtration to remove macromolecules more than 50 kD, digestion and aflatoxin extraction in Wang J S et al. (1996). Temporal Patterns of Aflatoxin-Albumin Adducts in Hepatitis B Surface Antigen-positive and Antigen-negative Residents of Daxin, Qidong County, People's Republic of China. Cancer Epidemiology, Biomarkers and Prevention, 5, 253-261. Wang J S et al. disclosed a method for high albumin recovery in <1 hour and minimized sample transfer. They use high-speed centrifugal and 50 kD-MW cutoff filtration units to concentrate human serum and rapidly isolate albumin from other proteins. But, in fact, it's hard to isolate albumin from other proteins with the 50 kD-MW cutoff filter unit since almost all the serum proteins are more than 50 kD MW. (See attached serum-protein-table from Norbert W. Tietz Textbook of Clinical Chemistry, p561, 1986). Besides, the recovered albumin was further digested with Pronase enzyme to obtain aflatoxin-polypeptide or aflatoxin-lysine adduct only. (See Cancer Epidemiology Biomarkers & Prevention, Vol.5, page 256, 2^{nd} column, Results) However, the process of Pronase digestion always needs >16 hrs. (See Sheabar et al. Carcinogenesis, Vol 14, pages 1204, 1993. FIG. 1.). The aforementioned references, Chen Chien-Jen et al. (1996). Elevated Aflatoxin Exposure and Increased Risk of Hepatocellular Carcinoma. Hepatology, 24(1), 38-42; Wang L Y et al., (1996) Alflatoxin Exposure and Risk of Hepatocellular Carcinoma in Taiwan. Int. J. Cancer, 67, 620-625; and Wang J S et al. (1996). Temporal Patterns of Aflatoxin-Albumin Adducts in Hepatitis B Surface Antigen-positive and Antigen-negative Residents of Daxin, Qidong County, People's Republic of China. Cancer Epidemiology, Biomarkers and Prevention, 5, 253-261 are included herein in their entirety.

This invention discloses one method which was used to test aflatoxin-albumin adduct in serum without the disadvantage of pretreatment. Besides, this invention also discloses a testing method to evaluate the extent of aflatoxin exposure by calculate the gram concentration ratio of aflatoxin B1-albumin adducts to albumin in serum, which is very useful to predict the future risk of liver cancer.

### SUMMARY OF THE INVENTION

It is therefore an object of the present invention to provide a more convenient method for testing aflatoxin-albumin adducts. Serum does not need to be pretreated with extraction or enzyme digestion prior to the test. As used herein, serum is the fluid portion of the blood obtained after removal of fibrin and blood cells. Once directly obtained from the human subject, the serum is used in this invention, as is, without any treatments similar to those described in the earlier references. For example, the serum does not require the addition of stabilizers, no dilution is necessary and no other proteins need to be removed from the serum for use in this application since the assay shows specificity and sensitivity. There is neither enhancement nor inhibition interferent in the serum matrix for this method.

It is therefore another object of the present invention to provide a method for evaluating the extent of a person's aflatoxin exposure, and to predict the risk of liver cancer. Further, the method shows the relationship of aflatoxin exposure and hepatocarcinoma.

### BRIEF DESCRIPTION OF THE DRAWINGS

FIG. 1 is a graph showing the method of competitive inhibition radioimmunometric assay.

FIG. 2 is a graph showing the radioimmunoassay testing kit for detection of aflatoxin-albumin adducts.

FIG. 3 is a graph showing there is no matrix effect in the dose response curve for testing of aflatoxin-albumin adducts.

FIG. 4 is a graph showing the specificity of primary antibody against aflatoxin-albumin adduct. It exists no cross-reactivity to albumin.

FIG. 5 is a graph showing the detection range of the radioimmunoassay testing kit in this invention.

### DETAILED DESCRIPTION OF THE PREFERRED EMBODIMENTS

In one embodiment, the present invention discloses a method for testing the presence of aflatoxin-albumin adducts in human serum comprising the following steps:

(a) providing a solid phase comprising a predetermined amount of aflatoxin-albumin adduct pre-applied to the solid phase and the aflatoxin-albumin adducts can be fixed with methods known to those skilled in the art;

(b) mixing a predetermined amount of a primary antibody specific for aflatoxin-albumin adduct with a serum sample such that the primary antibody and serum sample are in contact with the solid phase;

(c) incubating the primary antibody, sample serum and solid phase from step (b);

(d) washing the solid phase such that any primary antibody having bound to a aflatoxin-albumin adduct from the sample serum is removed and further such that any primary antibody having been bound to the solid phase aflatoxin-albumin adduct remains bound to the solid phase aflatoxin-albumin adduct; and

(e) adding a labeled secondary antibody such as I-125, labeled secondary antibody, or alkaline phosphatase, labeled secondary antibody, to the solid phase;

wherein the molar ratio of solid phase aflatoxin-albumin adduct to primary antibody is in the range of from 25 to 29.

The solid phase can be for example a microplate and the solid phase binds a predetermined amount of antigen, for example 20 μg/mL of aflatoxin-albumin adducts, which is not a limiting example. A mixture of a predetermined amount of primary antibody, a non-limiting example includes a rabbit antibody, and a sample of human serum or a standard is brought into contact with the solid phase. During incubation, which can last from about 30 minutes to about 16 hours, the solid phase aflatoxin-albumin adducts compete with the serum aflatoxin-albumin adducts for binding with the primary antibody. Thus, some of the primary antibody binds to the solid phase aflatoxin-albumin adduct and some of the primary antibody binds to the serum aflatoxin-albumin adduct leaving some of the solid phase aflatoxin-albumin adduct unbound. The labeled secondary antibody is added and will bind with only the primary antibody. If some of the primary antibody was washed away because it was bound to serum aflatoxin-albumin adduct, there will be measurably less labeled secondary antibody remaining. Otherwise, if there was no aflatoxin-albumin adduct in the serum, the primary antibody would be bound to measurably more solid phase aflatoxin-albumin adducts.

In another embodiment, the present invention discloses a solid phase microplate wherein the solid phase microplate binds about 20 μg/mL of aflatoxin-albumin adduct as an antigen.

In still another embodiment of the present invention, the primary antibody is a polyclonal antibody specific to aflatoxin-albumin, and the primary antibody is derived by repeatedly introducing aflatoxin-keyhole limpet hemocyanin (KLH) into a rabbit so that the polyclonal primary antibody are produced in blood serum of the rabbit.

In still yet another embodiment of the present invention, the secondary antibody is a polyclonal antibody specific to the primary antibody, and further the secondary antibody is derived by introducing a rabbit immunoglobulin into animals other than rabbits, so that the polyclonal antibody are provided in the blood serum of such other animal.

In one embodiment, the present invention discloses a method to determine aflatoxin exposure level in a human being comprising the following steps:

(a) providing a solid phase comprising a predetermined amount of aflatoxin-albumin adduct pre-applied to the solid phase;

(b) mixing a predetermined amount of a primary antibody specific for aflatoxin-albumin adduct with a serum sample such that the primary antibody and serum sample are in contact with the solid phase;

(c) incubating the primary antibody, sample serum and solid phase from step (b);

(d) washing the solid phase such that any primary antibody having bound to a aflatoxin-albumin adduct from the sample serum is removed and further such that any primary antibody having been bound to the solid phase aflatoxin-albumin adduct remains bound to the solid phase aflatoxin-albumin adduct;

(e) adding I-125 labeled secondary antibody or alkaline phospatase labeled secondary antibody to the solid phase; and

(f) the aflatoxin exposure measuring includes both testing of aflatoxin-albumin and albumin. The aflatoxin-albumin is measured by the above process, however, the albumin is measured by formation of an albumin bromocresol green complex, through the use of applied electromagnetic radiation with a wavelength of 628 nm. The level of aflatoxin exposure is determined by the concentration ratio of serum sample aflatoxin-albumin adduct (as detected by the present method) to albumin, expressed with the unit of ng aflatoxin-albumin adduct/mg albumin.

In still another embodiment, the present invention discloses a method for determining if a carcinogenic level of aflatoxin exposure has occurred. For example, if the level of aflatoxin exposure is more than the normal cutoff value of 0.532 ng aflatoxin-albumin adduct/mg albumin, then there will be a 7.97-fold higher risk to people to get hepatocarcinoma than to people with no aflatoxin exposure.

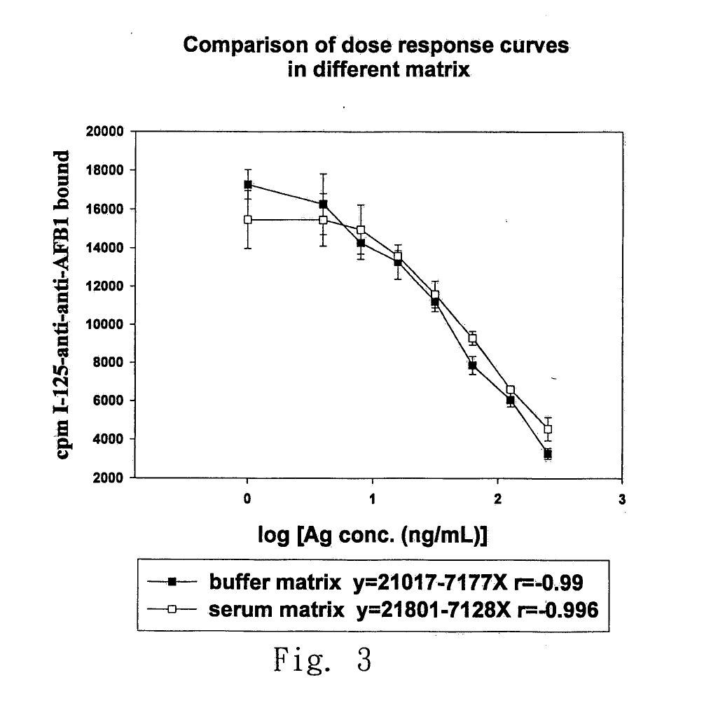

Further, the present invention relates to a novel method for detecting aflatoxin-albumin adduct as an antigen using a competitive inhibition radioimmunometric assay or enzyme-linked immunosorbent assay, the method comprising: providing primary antibody and testing serum on a microplate onto which aflatoxin-albumin adduct antigen is bound by techniques well known to practitioners skilled in the art, then washing away material unbound to the microplate after enough incubation, then using I-125-labelled secondary antibody or alkaline-phospatase labeled secondary antibody to detect the immune complex bound onto the microplate, wherein there are enough aflatoxin-albumin adducts bound on it, and both primary and secondary antibodies are polyclonal antibodies, derived through repeated introduction of aflatoxin-KLH into rabbits and by repeatedly introducing rabbit immunoglobulin into animals other than rabbit, respectively. The primary antibody has specificity for aflatoxin-albumin adducts, without substantial cross reactivity to albumin. The secondary antibody has specificity for rabbit immunoglobulin but minimal cross-reaction with immunoglobulin from bovine and human. FIG. 3 shows there is substantially no difference in dose response curves for aflatoxin-albumin adducts in serum matrix and buffer matrix. It indicates there is no interferent in serum materials to enhance or inhibit the immune complex formation by primary antibody and aflatoxin-albumin adducts.

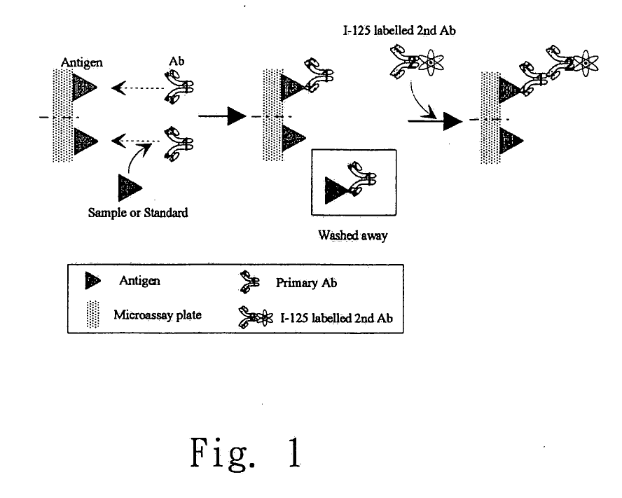

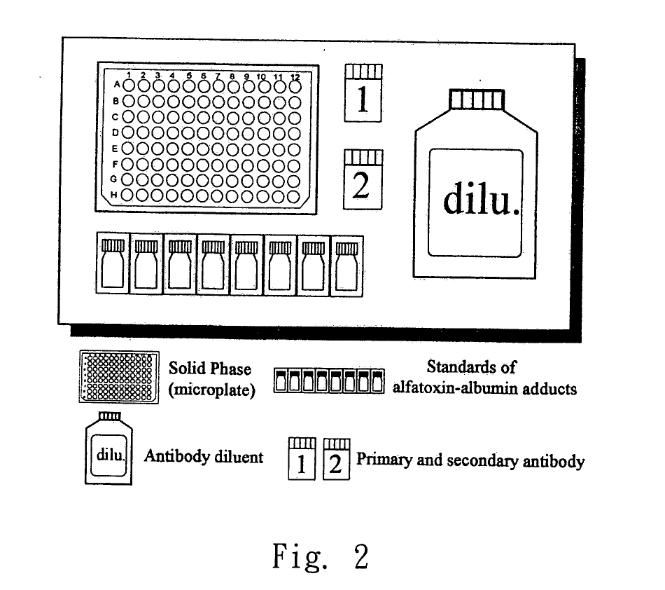

Finally the present invention relates to a method for evaluation of the dose of aflatoxin exposure, wherein aflatoxin-albumin testing only could not predict the risk of liver cancer, but testing for ng aflatoxin-albumin adducts per mg albumin is statistically clinically correlated with liver cancer, and wherein we set 0.532 ng aflatoxin-albumin adduct per mg albumin as normal cutoff value, and wherein there are 7.97 folds higher risk in people beyond this cutoff value than normal healthy persons, and wherein the aflatoxin-albumin adducts are testing by kit of FIG. 2 and method of FIG. 1, and wherein albumin is testing by dye-binding method which albumin and bromcresol green are allowed to bind at pH4.2 and absorption of the BCG-albumin complex is determined spectrophotometrically at 628 nm. At pH4.2, albumin acts as a cation to bind the anionic dye.

In this application, there are two ways to guarantee that the test serum does not have interfering substances and that any pretreatment of serum is unnecessary. In other words, it is unnecessary to remove albumin or other protein from sample serum or extract out the intended target aflatoxin-albumin adducts or purified aflatoxin from the sample serum.

First, the known amount of aflatoxin-albumin adducts, pure chemicals obtained from a pharmaceutical supplier, can be accurately and quantitatively measured after being added to the serum matrix. The accuracy of the aflatoxin-albumin adducts is measured by a recovery test. In the recovery experiment, the sample is divided into two aliquots. One aliquot is spiked with analyte. An equivalent amount of diluents is added to the second; this is the baseline sample. The two samples are then analyzed. The baseline sample provides the original amount of analyte. The difference between the spiked sample and the baseline sample indicates the amount of analyte “recovered”. The amount “added” is calculated from the concentration of the stock solution of the analyte and the volume added. The volume of analyte added to the sample was less than 10% to avoid major disruption of the sample matrix and pipetting accuracy is critical because the amount of added analyte is calculated from the volume. The calculation of recovery is defined as the ratio of the amount recovered to the amount added and is given as a percentage. If it existed any enhanced or inhibited interference, the all over recovery will be significantly different than 100%. In our recovery experiment, aflatoxin-albumin adducts are added to healthy normal serum not receiving aflatoxin expose. The average recovery is 100.9±14.7%, data shown in Table 1 hereunder, which means that no interference existed in the serum when analyzed by our radioimmunoassay (RIA) kit and system.

Second, in an RIA procedure, the validity of the assay requires that the immunochemical behavior of standards and the unknowns be identical. In order to accurately determined the concentration of an unknown, it must react immunologically identically to the standards, although it may be structurally dissimilar. Parallelism test is shown in FIG. 3. Parallelism in a RIA is demonstrated by diluting elevated unknowns the same way a standard curve would be prepared, and then comparing the dilutions to curves obtained with the pure standard. If the two curves are identical or parallel, the antibody is immunologically recognizing the same structure in the unknown as it is in the standard. When diluting unknowns for parallelism studies or for routine assays it is important to recognize that the analyte concentration may be critical and should not vary between standards and unknowns. On a more practical level, parallelism is used to determine linearity or dilution effects. For example, if one has an elevated serum aflatoxin-albumin with RIA testing, could the serum be diluted twofold or tenfold without disturbing the assay equilibrium? Failure to confirm parallel behavior in a RIA could mean that cross-reacting substances are present in the sample. This may preclude use of the assay kit, or necessitate sample purification or preparation steps prior to RIA. These pre-RIA preparation steps will only increase the total assay error. FIG. 3 shows that the diluting curve of elevated unknowns in the linear range from 15.5 ng/mL to 125 ng/mL is parallel to the standard curve prepared, which indicates that there is no matrix effect in the dose response curves for testing of aflatoxin-albumin adducts.

Sample Protocol

In one aspect, the present invention can follow this sample protocol:
1—solid 96 well plates precoated with aflatoxin-albumin
2—apply sample (serum, standard) and primary antibody specific for aflatoxin-albumin into the solid 96 well plates and incubate for 4 hours at room temperature
3—wash the above solid 96-well plate with TTBS (0.05% Tween 20, 10 mM Tris buffer, pH7.8, 0.15M NaCl)
4—apply I-125 labeled secondary antibody specific to rabbit immunoglobulin into the above solid 96-well plate and incubate for 1 hour at room temperature
5—wash the above solid 96-well plate with TTBS (0.05% Tween 20, 10 mM Tris buffer, pH7.8, 0.15M NaCl)
6—quantitate the γ-count of I-125 labeled secondary antibody in the above solid 96-well plate by γ-counter
7—a standard curve with concentration-response (γ count) relationship was constructed from standard using aflatoxin-albumin at concentration of 0-250 ng/mL
8—the aflatoxin-albumin concentration in serum sample is calculated based on the constructed standard curve
9—method validation with the recovery test

### EXAMPLE 1

### Dose Response Curves of Aflatoxin-albumin Adducts in Different Matrix

Aflatoxin-albumin adduct standards in buffer matrix or serum matrix are adding to microplate respectively, wherein their standard range is from 0 to 250 ng/mL. Following the steps of FIG. 1, both of them are incubated with specific primary antibody, after enough time, washing away the unbound materials on the microplate. The immune complex bound to microplate was determined by I-125-labelled secondary antibody. As FIG. 3 indicates, there seems to be no differences between standards in buffer matrix and those in serum matrix, because of their parallel dose response curves. The results indicate there is no interferent in serum matrix for aflatoxin-albumin testing. So the pretreatment steps of serum, such as extraction or enzyme digestion, is unnecessary.

### EXAMPLE 2

### Study on Specificity of Primary Antibody

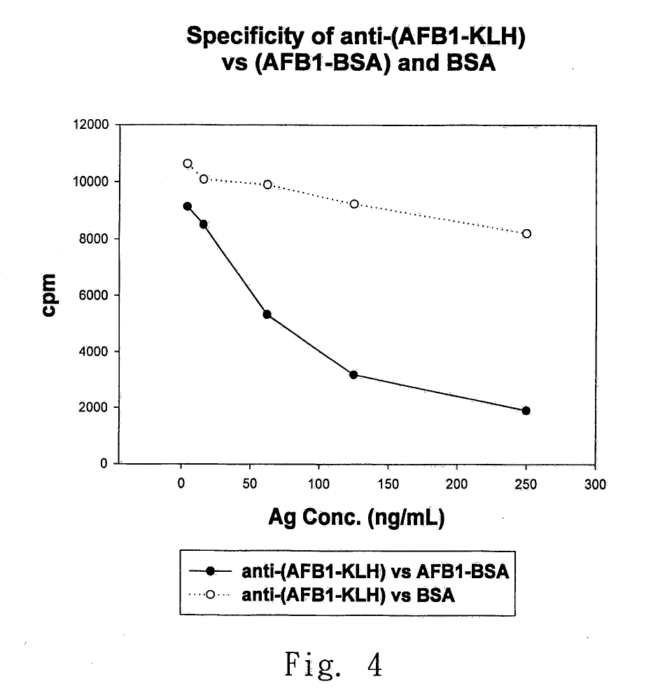

Particular aliquots of 0-250 ng/mL standards of aflatoxin-albumin adducts or albumin is adding to microplate, respectively. Following the steps of 1, both of them are incubated with specific primary antibody, after enough time, washing away the unbound materials on the microplate. The immune complex bound to microplate was quantified by I-125-labelled secondary antibody. FIG. 4 shows two dose response curves for aflatoxin-albumin adduct and albumin as antigens, respectively. There is no 50% inhibition of cross-reaction in the dose response curve of albumin as antigen, which indicates the primary antibody has specificity to aflatoxin-albumin adducts but has no cross-reaction with albumin.

### EXAMPLE 3

### Study on Detection Range of Aflatoxin-albumin Radioimmunoassay Testing Kit

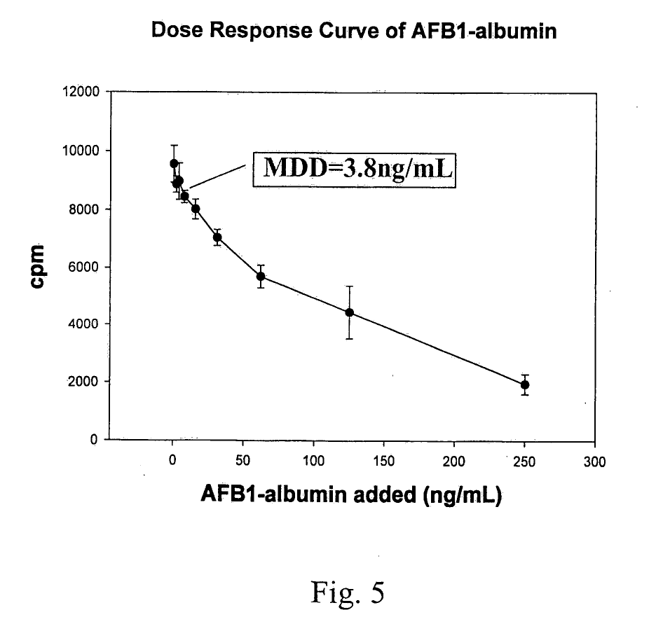

0-250 ng/mL standards of aflatoxin-albumin adducts, as standards in FIG. 2, are adding to microplate in predetermined amounts. Following the steps of FIG. 1 by incubation enough time with specific primary antibody, washing away the unbound material. The bound immune complex were quantified by I-125-labeled secondary antibody. FIG. 5 shows the performance of dose response curve by this kit. In this curve, the detection limit is 3.8 ng/mL, which is defined as the smallest concentration of an antigen that could be statistically distinguished from a zero level in an assay. The detection range is from 3.8 ng/mL to 250 ng/mL (FIG. 5) and there is linearity from 15.5 ng/mL to 125 ng/mL.

### EXAMPLE 4

### Study on Accuracy of Aflatoxin-albumin Radioimmunoassay Testing Kit

The accuracy is measured by the recovery of known concentration of aflatoxin-albumin adducts in serum matrix. Following the steps of FIG. 1, we got the detected concentration by comparing their cpms with the standard curve. The concentration added in the serum sample is 7.7 ng/mL and 31 ng/mL, respectively, and the concentrations recovered are 8.3±1.4 ng/mL and 29.7±3.6 ng/mL, respectively. The recovery, which is called as accuracy and often calculated by concentration recovered divided by concentration added, is 100.9±14%.

TABLE 1
The accuracy of this aflatoxin-albumin
adducts radioimmunoassay testing kit.

| | | |
|---|---|---|
| AFB1-albumin | AFB1-albumin | recovery |
| added | measured | (%) |

| | | | |
|---|---|---|---|
| 31 ng/mL | 35 | ng/mL | 113 |
| | 28 | ng/mL | 90 |
| | 28.8 | ng/mL | 93 |
| | 27 | ng/mL | 87 |
| 7.7 ng/mL | 8.6 | ng/mL | 112 |
| | 6.8 | ng/mL | 88 |
| | 9.5 | ng/mL | 123 |
| | | | 100.9 ± 14.7% |

### EXAMPLE 5

### Case-control Study for Aflatoxin-albumin Testing

Choose two groups for case-control study. Both of their age mean were 53. Classified as “normals”, 16 people were identified as having normal albumin, GOT (glutamate oxaloacetic transaminase), GPT (glutamate pyruvate transaminase), abdominal sonography, α-fetoprotein and no history of hepatoma. Classified as “hepatoma”, 16 patients were identified as having primary HCC (hepatocellular carcinoma) histologically confirmed by needle biopsy. All of the samples were testing for aflatoxin-albumin adducts by using kit of FIG. 2 and method of FIG. 1. The 95% confidence intervals of testing are 6.8˜12.6 ng/mL for “normal” and 10.7˜71.3 ng/mL for “hepatoma”, respectively. There is some overlap each other. See Table 2. But if testing for ng aflatoxin-albumin adduct per mg albumin, which albumin is tested by 628 nm absorption by complex with BCG, the 95% confidence intervals of testing are 0.14˜0.28 ng/mg for “normal” and 0.29˜1.97 ng/mg for “hepatoma”, respectively (Table 3). There is statistically significant difference each other. It indicates the high correlation of aflatoxin exposure and liver cancer.

The present invention indicates a method for quantification of aflatoxin-albumin per mg albumin in sample could be used to evaluate the dose of aflatoxin exposure, which is highly correlated to liver cancer.

TABLE 2
Testing of aflatoxin-albumin adducts
in 16 Normals and 16 Hepatomas

| | Normals | Hepatomas |
|---|---|---|
| | (ng/mL) | (ng/mL) |
| Mean | 9.7 | 41 |
| Mean of 95% confidence interval | 6.8˜12.6 | 10.7˜71.3 |

TABLE 3
Testing of ng aflatoxin-albumin adducts per
mg albumin in 16 Normals and 16 Hepatomas

| | Normals | Hepatomas |
|---|---|---|
| | (×10⁻¹, ng/mg) | (×10⁻¹, ng/mg) |
| Mean | 2.1 | 11.3 |
| Mean of 95% confidence interval | 1.4˜2.8 | 2.9˜19.7 |

### EXAMPLE 6

### Normal Cutoff Value for Dose of Aflatoxin Exposure

To validate the normal cutoff value for dose evaluation of aflatoxin exposure, 195 patients were random chosen for testing of ng aflatoxin-albumin per mg albumin in serum. Classified as “normals”, 99 people were identified as having normal albumin, GOT, GPT, abdominal sonography, α-fetoprotein and no history of hepatoma. Classified as “hepatoma”, 96 patients were identified as having primary HCC histologically confirmed by needle biopsy.

All of these samples were tested for ng aflatoxin-albumin per mg albumin in serum, which aflatoxin-albumin was tested by using kit of FIG. 2 and method of FIG. 1, and albumin was tested by absorption at 628 nm by complex with BCG. The 95% confidence intervals of ng aflatoxin-albumin per mg albumin testing are 0.39˜0.532 ng/mg and 1.1˜1.7 ng/mg, respectively, which confirms again the high correlation of aflatoxin exposure and liver cancer, in addition, the normal cutoff value is 0.532 ng/mg (see Table 4).

TABLE 4
Testing of ng aflatoxin-albumin adducts per
mg albumin in 99 Normals and 96 Hepatomas

| | Normals | Hepatomas |
|---|---|---|
| | (×10⁻¹, ng/mg) | (×10⁻¹, ng/mg) |
| Mean | 4.6 | 14.0 |
| Mean of 95% confidence interval | 3.9˜5.32 | 11˜17 |

### EXAMPLE 7

### Risk of Liver Cancer if Dose of Aflatoxin Exposure is More Than 5.32 ng/mg

The same 195 study identified a group of 96 liver cancer cases, ages about 55, and a group of 99 age, sex, resident-matched healthy controls by random chosen. The main purpose of the study was to look at the effect of aflatoxin exposure on liver cancer risk. In this study, aflatoxin exposure “yes” was defined as exposure dose more than normal cutoff value. To display the data, a 2×2 table relating case-control status to aflatoxin exposure can be constructed for “hepatomas” and “controls”. The data are given in Table 5.

TABLE 5
Risk of liver cancer on aflatoxin exposure

disease

| exposure | Hepatomas | Normals | |
|---|---|---|---|
| yes | 70 | 25 | 95 |
| no | 26 | 74 | 100 |
| | 96 | 99 | 195 |

The odds ratio is 7.97 (4.2˜15.1).

In this case-control study, there are 70 people among 96 “hepatomas” aflatoxin exposure “yes”, but there are 25 people among 99 healthy “controls” aflatoxin exposure “yes”. The estimation of odds ratio was according to the Mantel-Haenzel's method. Based on Table 5, the odds ratio OR is =(70×74)/(25×26)=7.97, and its 95% confidence interval is 7.97×exp[±1.96√(1/70+1/74+1/26+1/25)]=4.2˜15.1

The risk to have the liver cancer is 7.97 measured by odds ratio for people with aflatoxin exposure, which is to say 7.97-fold higher risk to get hepatocarcinoma than people with no aflatoxin exposure.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A method for testing the presence of aflatoxin-albumin adducts in human serum comprising the following steps:
(a) providing a solid phase comprising a predetermined amount of aflatoxin-albumin adduct pre-applied to the solid phase;
(b) mixing a predetermined amount of a primary antibody specific for the aflatoxin-albumin adduct with a serum sample such that the primary antibody and the serum sample are in contact with the solid phase;
(c) incubating the primary antibody, sample serum and the solid phase from step (b);
(d) washing the solid phase such that any primary antibody having bound to said aflatoxin-albumin adduct from the sample serum is removed and further such that any primary antibody having been bound to the solid phase aflatoxin-albumin adduct remains bound to the solid phase aflatoxin-albumin adduct; and
(e) adding a I-125 labeled secondary antibody or an alkaline phosphatase labeled secondary antibody to the solid phase;
wherein a molar ratio of the solid phase aflatoxin-albumin adduct to the primary antibody is in the range of from 25 to 29.

2. The method of claim 1, wherein the solid phase is a microplate.

3. The method of claim 1, wherein the solid phase is a solid phase microplate and further wherein the solid phase microplate binds about 20 μg/mL of aflatoxin-albumin adduct as an antigen.

4. The method of claim 1, wherein the primary antibody is a polyclonal antibody specific to aflatoxin-albumin, and wherein the primary antibody is derived by repeatedly introducing an aflatoxin-KLH into a rabbit so that the polyclonal primary antibody is produced in a blood serum of the rabbit.

5. The method of claim 1, wherein the secondary antibody is a polyclonal antibody specific to the primary antibody, and further wherein the secondary antibody is derived by introducing a rabbit immunoglobulin into an animal other than the rabbit, so that the polyclonal antibody is provided in the blood serum of such other animal.

6. A method for determining a carcinogenic level of aflatoxin exposure in a human being comprising the following steps:
(a) providing a solid phase comprising a predetermined amount of an aflatoxin-albumin adduct pre-applied to the solid phase;
(b) mixing a predetermined amount of a primary antibody specific for the aflatoxin-albumin adduct with a serum sample such that the primary antibody and serum sample are in contact with the solid phase;
(c) incubating the primary antibody, sample serum and solid phase from step (b);
(d) washing the solid phase such that any primary antibody having bound to the aflatoxin-albumin adduct from the sample serum is removed and further such that any primary antibody having been bound to the solid phase aflatoxin-albumin adduct remains bound to the solid phase aflatoxin-albumin adduct;
(e) adding a I-125 labeled secondary antibody or an alkaline phosphatase labeled secondary antibody to the solid phase; and
(f) determining the level of aflatoxin exposure by calculating a concentration ratio of the serum sample aflatoxin-albumin adduct to the serum sample albumin, which is expressed with a unit of ng aflatoxin-albumin adduct/mg albumin and where the aflatoxin-albumin is measured by the process of claim 1; however, the albumin is measured by formation of an albumin bromocresol green complex, through the use of applied electromagnetic radiation With a wavelength of 628 nm.

7. The method for determining if a carcinogenic level of aflatoxin exposure has occurred in claim 6, wherein if the level of aflatoxin exposure is more than a normal cutoff value of 0.532 ng aflatoxin-albumin adduct/mg albumin, there will be a 7.97-fold higher risk for a person to contract hepatocarcinoma than in the same person with no aflatoxin exposure.
